(19) United States
(12) Patent Application Publication
(10) Pub. No.: US 2008/0287976 A1
(43) Pub. Date: Nov. 20, 2008

(54) Gastric band with engagement member

(76) Inventors: Lauren S. Weaner, Beavercreek, OH (US); Jeffrey P. Wiley, Milford, OH (US); Kristin L. Jambor, Cincinnati, OH (US); Christopher W. Widenhouse, Clarksville, OH (US); Patrick J. Swindon, Cincinnati, OH (US)

Correspondence Address: WELSH & FLAXMAN LLC, 2000 DUKE STREET, SUITE 100, ALEXANDRIA, VA, 22314, US

(21) Appl. No.: 11/798,501

(22) Filed: May 14, 2007

(51) Int. Cl. A61B 17/08

(52) U.S. Cl. 606/157

(57) ABSTRACT

A gastric band includes a gastric band body having a first end and second end. The first end and the second end are provided with a respective first latching member and a second latching member. The first end includes a tip and an engagement member proximal the first latching member such that the engagement member is on a side of the first latching member opposite the tip. The engagement member enhances one's ability to grasp the first end of the gastric band body during application and removal.

### BACKGROUND OF THE INVENTION

1. Field of the Invention

The invention relates to a gastric band and related accessories. More particularly, the invention relates to a gastric band including an engagement member enhancing manipulation thereof during application and removal of a gastric band about a stomach.

2. Description of the Related Art

Morbid obesity is a serious medical condition. In fact, morbid obesity has become highly pervasive in the United States, as well as other countries, and the trend appears to be heading in a negative direction. Complications associated with morbid obesity include hypertension, diabetes, coronary artery disease, stroke, congestive heart failure, multiple orthopedic problems and pulmonary insufficiency with markedly decreased life expectancy. With this in mind, and as those skilled in the art will certainly appreciate, the monetary and physical costs associated with morbid obesity are substantial. In fact, it is estimated the costs relating to obesity are in excess of one hundred billion dollars in the United States alone.

A variety of surgical procedures have been developed to treat obesity. The most common currently performed procedure is Roux-en-Y gastric bypass (RYGB). This procedure is highly complex and is commonly utilized to treat people exhibiting morbid obesity. Other forms of bariatric surgery include Fobi pouch, bilio-pancreatic diversion, and gastroplastic or “stomach stapling”. In addition, implantable devices are known which limit the passage of food through the stomach and affect satiety.

In view of the highly invasive nature of many of these procedures, efforts have been made to develop less traumatic and less invasive procedures. Gastric-banding is one of these methods. Gastric-banding is a type of gastric reduction surgery attempting to limit food intake by reducing the size of the stomach. In contrast to RYGB and other stomach reduction procedures, gastric-banding does not require the alteration of the anatomy of the digestive tract in the duodenum or jejunum.

Since the early 1980's, gastric bands have provided an effective alternative to gastric bypass and other irreversible surgical weight loss treatments for the morbidly obese. Several alternate procedures are performed under the heading of gastric-banding. Some banding techniques employ a gastric ring, others use a band, some use stomach staples and still other procedures use a combination of rings, bands and staples. Among the procedures most commonly performed are vertical banded gastroplasty (VBG), silastic ring gastroplasty (SRG) and adjustable silastic gastric banding (AGB).

In general, the gastric band is wrapped around an upper portion of the patient's stomach, forming a stoma that is less than the normal interior diameter of the stomach. This restricts food passing from an upper portion to a lower digestive portion of the stomach. When the stoma is of an appropriate size, food held in the upper portion of the stomach provides a feeling of fullness that discourages overeating.

More particularly, and in practice, the gastric band is inserted behind the stomach and the ends of the gastric band are coupled to latch the device about the stomach. However, it is often difficult to manipulate the gastric band during application. As such, the present gastric band has been developed in an effort to alleviate these problems.

### SUMMARY OF THE INVENTION

It is, therefore, an object of the present invention to provide a gastric band, having a gastric band body with a first end and second end, the first end and the second end being provided with a respective first latching member and a second latching member, the first end including a tip and an engagement member proximal the first latching member such that the engagement member is on a side of the first latching member opposite the tip.

Another object of the present invention is to form the engagement member as a thru-hole formed in the gastric band body and the thru-hole passes laterally through the gastric band body. Further, the thru-hole includes a top surface and a bottom surface, and the top surface is narrower in its lateral dimension than the bottom surface.

Still another object of the present invention is to form the engagement member as a shell-like member protruding from the gastric band body, wherein the shell-like member includes a convex outer surface with a first end adjacent the first latching member and a second end facing away from the first latching member, and the second end of the shell-like member is open providing an access opening to a cavity of the shell-like member.

Yet another object of the present invention is to form the engagement member as a thickened region with a recess formed therein.

Another object of the present invention is to form the engagement member as a grasping pocket, wherein the grasping pocket is formed in a thickened region in the gastric band body and the grasping pocket includes an internal cavity with an access opening. Further, the access opening substantially faces away from the first latching member.

Still another object of the present invention is to form the engagement member as a raised, necked-in region, wherein the necked-in region includes a generally narrowed section and the narrowed section includes an hourglass shape.

Yet another object of the present invention is to form the engagement member as a lateral thru-hole with a center hole extending through a top surface thereof.

A further object of the present invention is to form the engagement member as a necked-in region wherein lateral walls of the gastric band body are closer together in the necked-in region along the top surface thereof than along the bottom surface of the gastric band body.

It is also an object of the present invention to raise the necked-in region relative the top surface of a remainder of the gastric band body.

Other objects and advantages of the present invention will become apparent from the following detailed description when viewed in conjunction with the accompanying drawings, which set forth certain embodiments of the invention.

### BRIEF DESCRIPTION OF THE DRAWINGS

FIG. 1 shows a gastric band in accordance with a first embodiment of the present invention positioned around a stomach.

FIGS. 2 and 3 respectively show a detailed perspective view and a cross sectional view of the first end of the gastric band shown in FIG. 1.

FIGS. 4 and 5 respectively show a perspective view and a cross sectional view of the first end of a gastric band in accordance with a second embodiment.

FIGS. 6 and 7 respectively show a perspective view and a cross sectional view of the first end of a gastric band in accordance with a third embodiment.

FIGS. 8 and 9 respectively show a perspective view and a cross sectional view of the first end of a gastric band in accordance with a fourth embodiment.

FIGS. 10 and 11 respectively show a perspective view and a cross sectional view of the first end of a gastric band in accordance with a fifth embodiment.

FIGS. 12 and 13 respectively show a perspective view and a cross sectional view of the first end of a gastric band in accordance with a sixth embodiment.

FIGS. 14 and 15 respectively show a perspective view and a cross sectional view of the first end of a gastric band in accordance with a seventh embodiment.

### DESCRIPTION OF THE PREFERRED EMBODIMENTS

The detailed embodiments of the present invention are disclosed herein. It should be understood, however, that the disclosed embodiments are merely exemplary of the invention, which may be embodied in various forms. Therefore, the details disclosed herein are not to be interpreted as limiting, but merely as the basis for teaching one skilled in the art how to make and/or use the invention.

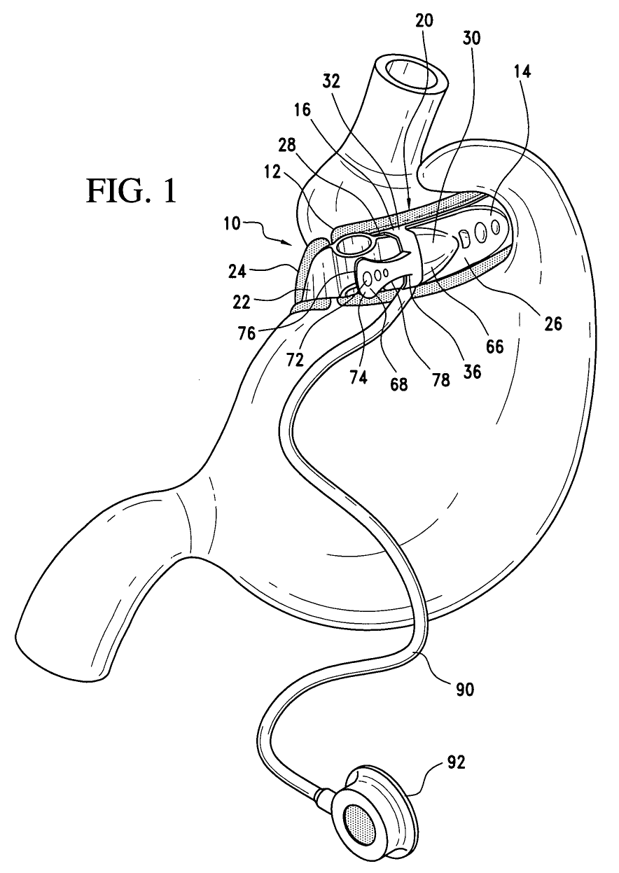

With reference to FIGS. 1, 2 and 3, a gastric band 10 in accordance with a preferred embodiment of the present invention includes a band body 12 having a first end 14 and an opposite second end 16. A latching mechanism 20 composed of a shell or a first latching member 30 and a collar or a second latching member 32 is secured to the respective ends of the band body 12 in a manner permitting selective attachment of the gastric band 10 about a patient's stomach. The band body 12 and latching mechanism 20 are preferably manufactured from silicone. Although, and as will be discussed below in greater detail, the gastric band 10 is a balloon-type gastric band, the present latching mechanism 20 may be used in conjunction with a variety of band structures without departing from the spirit of the present invention.

The gastric band 10 is shaped and dimensioned to circumscribe the stomach at a predetermined location reducing the size of the stomach. The gastric band 10 employs a flexible latching mechanism 20 capable of locking and unlocking without destruction of the latching mechanism 20 or significant reduction in retention capabilities after re-locking. The first and second ends 14, 16 respectively include a shell member 30 and a collar member 32 which act as both male and female members depending on the direction of motion and intent to lock or unlock the latching mechanism 20 of the present gastric band 10.

Referring to FIGS. 1 to 3, and in accordance with a preferred embodiment of the present invention, the gastric band 10, in particular, the gastric band body 12, is generally composed of a reinforcing belt 22 to which an elongated balloon 24 is secured. The belt 22 includes a first end 26 and a second end 28, which generally correspond to the first end 14 and the second end 16 of the gastric band body 12, to which shell member 30 and collar member 32 of the latching mechanism 20 are respectively secured. In accordance with a preferred embodiment, the latching members are the same as disclosed in commonly owned U.S. patent application Ser. No. 11/182,072, entitled “Latching Device for Gastric Band”, filed Jul. 15, 2005, which is incorporated herein by reference.

As those skilled in the art will certainly appreciate, the belt 22 includes an inner surface and an outer surface. The outer surface is substantially smooth and forms a substantial portion of the outer surface of the gastric band when it is secured about a patient's stomach. The inner surface of the belt 22 is shaped and dimensioned for attachment to the outer surface of the balloon 24.

With regard to the balloon 24, it also includes a first end, a second end, an inner surface and an outer surface. The inner surface is substantially smooth and is shaped and dimensioned for engaging the patient's stomach when the gastric band is secured thereto. The outer surface of the balloon 24 is shaped and dimensioned for coupling with the inner surface of the belt.

In accordance with a preferred embodiment, the basic construction of the balloon and belt is substantially as disclosed in commonly owned U.S. patent application Ser. Nos. 11/364,362, entitled “Gastric Band”, filed Mar. 1, 2006, and 11/364,363, entitled “Precurved Gastric Band”, filed Mar. 1, 2006, which are incorporated herein by reference.

As briefly mentioned above, the first end 14 of the gastric band 10 includes the shell member 30. The shell member 30 is generally composed of a hollow, half-moon shaped shell with a tab 34 for gripping and pulling through a collar member 32 composed of a semi-circular shaped aperture 36 on the second end 16 of the gastric band 10. The half-moon shaped shell of the shell member 30 collapses as it is pulled or pushed through the collar member 32 by a grasper. The collar member 32 includes a tongue such that the shell member 30 slides through the semi-circular shaped aperture 36 and under the tongue (not shown) during latching. Once the shell member 30 passes the tongue, the roles change. The first end 14 of the gastric band 10 functions as a female component when the shell member 30 resiliently returns to its original shape and is allowed to slide back onto the second end 16 (now a male component) and over the tongue. As such, the shell member 30 functions as both a male component and female component during operation of the latching mechanism 20 and the collar member 32 functions as both a male component and female component during operation of the latching mechanism 20; that is, the shell member 30 functions as a male component during insertion through the collar member 32 and a female component thereafter when the tongue is seated therein. Unlocking is achieved by employing graspers to pull the first end 14 forward away from the second end 16 removing the tongue from the shell member 30. The shape of the shell member 30 permits it to collapse and move under the tongue and through the collar member 32.

More particularly, the shell member 30 at the first end 14 of the gastric band 10 is generally a half-moon shaped shell with an open, wide end 40 tapering toward a narrow end 42 adjacent the tip 44 of the first end 14. The shell member 30 is substantially hollow and is formed from a material, for example, silicone, which permits compression and expansion thereof.

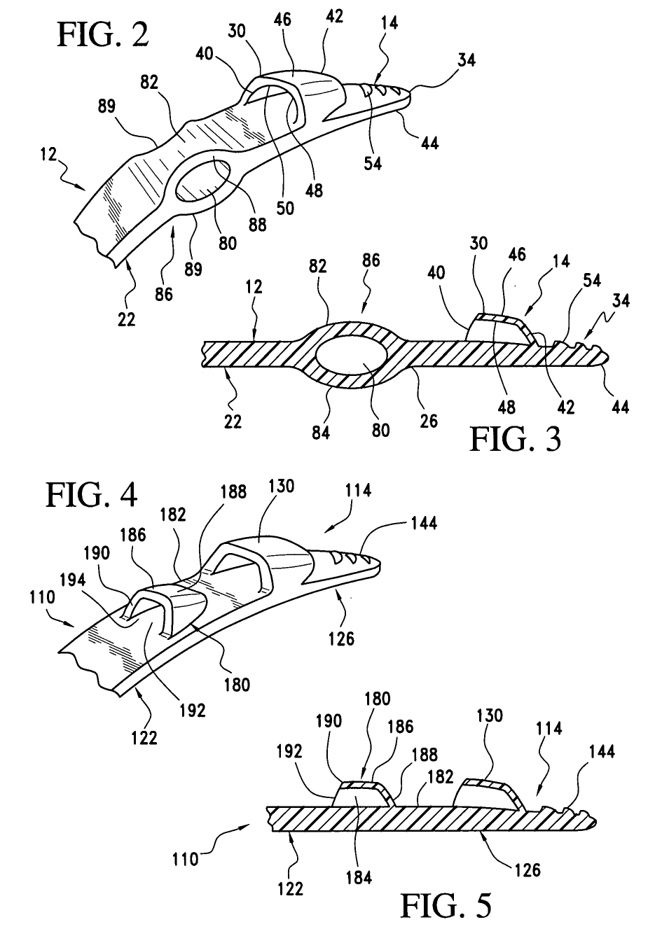

Referring to FIGS. 2 and 3, the shell member 30 is formed with a substantially semi-circular outer surface 46 when viewed from the wide end 40 thereof. The inner surface 48 of the shell member 30 adjacent the wide end 40 similarly has a substantially smooth semi-circular profile.

The shell member 30 is compressed and slid through the collar member 32 as discussed above. Thereafter, the center 50 of the wide end 40 returns to its original shape and fits over the tongue. When the gastric band 10 is unlatched, the shell member 30 is pulled forward away from the collar member 32 and the shell member 30 permits it to move under the tongue and through the collar member 32. The preformed shape of the shell member 30 not only acts as a guiding feature for the tongue to slide over the shell member 30 during unlocking, but will also allow the shell member 30 to more easily slide back through the aperture 36 of the collar member 32.

The tab 34 is formed with protrusions 54 assisting in grabbing the tab 34 during locking and unlocking.

Secure fastening of the shell member 30 with the collar member 32 is achieved by ensuring that after the shell member 30 compresses while passing through the collar member 32, the shell member 30 returns to its original shape and the wide end 40 of the shell member 30 abuts with the first edge 66 of the collar member 32.

Latching is further enhanced by providing the collar member 32 with a tongue extending from the collar member 32 away from the tip of the second end 16. The tongue is shaped and dimensioned to seat within the wide end 40 of the shell member 30 after the shell member 30 has passed through the collar member 32 and the gastric band 10 is tensioned as the first and second ends 14, 16 are drawn toward each other with the shell member 30 straining to move back through the collar member 32 toward an unlatched positioned. With this in mind, the tongue may be downwardly oriented such that it slides with the shell member 30 in a convenient and reliable manner. The tongue may be distinctly colored to provided an indication as to whether the latching mechanism 20 is properly locked.

Gripping of the second end 16 is further enhanced through the provision of a forward facing gripping member 68, that is, a gripping member facing the tip of the second end 16. The forward facing gripping member 68 is shaped and dimensioned to permit dual directional access for locking and unlocking of the latching mechanism 20. More particularly, the gripping member 68 includes protrusions 72 along the top and bottom surfaces 74, 76 thereof. These protrusions 72 facilitate gripping thereof along a first directional orientation. The gripping member 68 is further formed with an “hourglass” shape having a reinforced central section 78. The reinforced central section 78 allows for gripping in a second directional orientation.

The gripping member 68 is shaped and dimensioned to receive and center the shell member 30 as it passes through the collar member 32. The gripping member 68 also assists in compressing the shell member 30 as it passes through the collar member 32.

One of the objects of the present gastric band is an improvement to the gastric band's ease of use. With this in mind, various embodiments including an engagement member have been developed facilitating improved access to the first end of the gastric band for grasping during application and removal.

In particular, and with reference to FIGS. 1, 2 and 3, a gastric band 10 is disclosed wherein an engagement member in the form of a thru-hole 80 is provided in the belt 22 just proximal of the shell member 30 at the first end 14 of the gastric band 10, in particular, at the first end 26 of the belt 22 on the side of the shell member 30 opposite the tip 44 of the first end 14 of the gastric band 10. The thru-hole 80 passes laterally through the belt 22 adjacent the first end 26 thereof. As such, the thru-hole 80 includes a longitudinal axis that is substantially perpendicular to the longitudinal axis of the gastric band 10 when stretched in a substantially flat orientation.

The thru-hole 80 is shaped and dimensioned to allow a surgeon to slide a grasper in and manipulate the gastric band 10 from the side. In addition, the belt 22 region above the thru-hole 80 is tapered inward so that the surgeon can grasp the gastric band 10 from straight above. More particularly, the thru-hole 80 is defined by a top surface 82 and a bottom surface 84, and the top surface 82 is narrower in its lateral dimension (that is, as it extends from a first edge 88 of the belt 22 to the second edge 89 of the belt 22) than the bottom surface 84. As a result, if the surgeon comes straight onto this thru-hole region 86 of the belt 22 with a grasper, the grasper will bottom out on the belt 22 at the bottom surface 84, thereby protecting the balloon 24.

Referring to FIGS. 4 and 5, an alternate embodiment is disclosed. In accordance with this embodiment, an engagement member in the form of a secondary shell-like member 180 protrudes just proximal of the shell member 130 along the belt 122, in particular, at the first end 126 of the belt 122 on the side of the shell member 130 opposite the tip 144 of the first end 114 of the gastric band 110. The shell-like member 180 protrudes from the top surface 182 of the belt 122 allowing a surgeon access thereto. The shell-like member 180 includes a convex outer surface 186 with a first end 188 adjacent the shell member 130 and a second end 190 facing away from the shell member 130. The first end 188 of the shell-like member 180 is preferably closed, while the second end 190 of the shell-like member 130 is open providing an access opening 192 to the cavity 194 defined by the shell-like member 180. In practice, the shell-like member 180 is shaped and dimensioned such that a grasper may be fit inside the access opening 192 of the shell-like member 180, improving a surgeon's ability to manipulate the gastric band 110. Since the shell-like member 180 sits on a top surface 182 of the belt 122, the balloon 124 is once again protected from potential damage during manipulation.

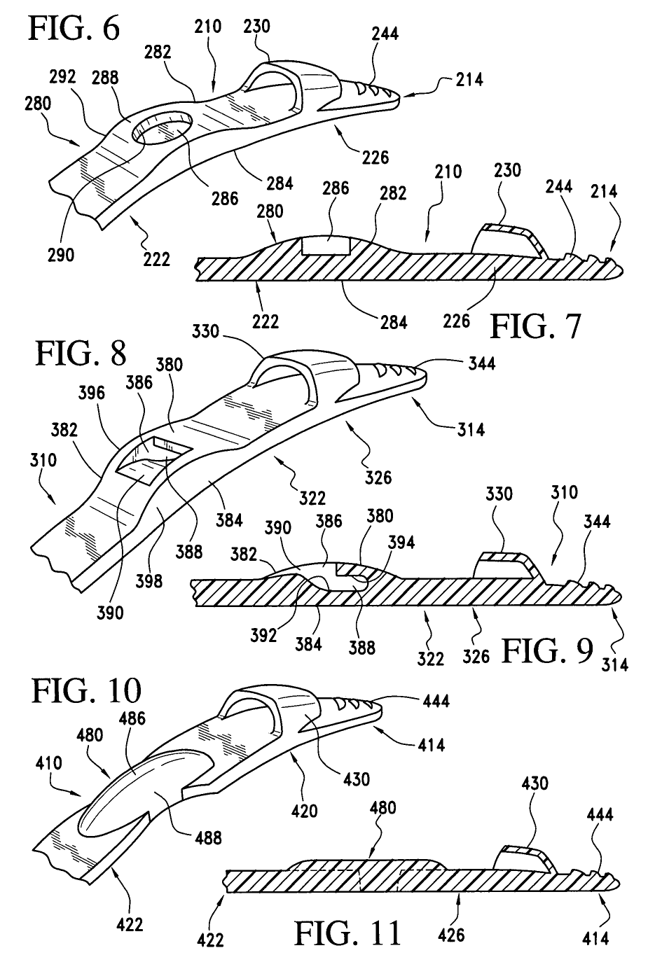

In accordance with yet a further embodiment, and with reference to FIGS. 6 and 7, the belt 222 of the gastric band 210 is provided with an engagement member in the form of a thickened region 280, that is, an area where the distance from the top surface 282 of the belt 222 to the bottom surface 284 of the belt 222 is greater than along other portions of the belt 222, just proximal of the shell member 230, in particular, at the first end 226 of the belt 222 on the side of the shell member 230 opposite the tip 244 of the first end 214 of the gastric band 210. The thickened region 280 provides an area for grasping by a surgeon when unlocking the shell member 230 from the collar member 232. A recess 286 is provided along the top surface 282 of the belt 222 in the thickened region 280. The recess 286 allows the surgeon to grab a thin region 288 on either side of the recess 286, which is defined by the inner wall 290 of the recess 286 and an outer edge 292 of the belt 222, for manipulation of the gastric band 210. Since the recess 288 is not a thru-hole, the belt 222 material itself protects the balloon 224.

With reference to yet a further embodiment, as shown in FIGS. 8 and 9, the belt 322 of the gastric band 310 is provided with an engagement member in the form of a grasping pocket 386 just proximal of the shell member 330, in particular, at the first end 326 of the belt 322 on the side of the shell member 330 opposite the tip 344 of the first end 314 of the gastric band 310. In accordance with this embodiment, the region just proximal of the shell member 330 is thickened as it extends between the top surface 382 and the bottom surface 384 of the belt 322 to define a thickened region 380. Within the thickened region 380 the grasping pocket 386 is provided. The grasping pocket 386 is shaped and dimensioned for placement of a grasper therein for manipulation of the gastric band 310. More particularly, the grasping pocket 386 includes an internal cavity 388 with an access opening 390 substantially facing away from the shell member 330. The internal cavity 388 is defined by a bottom wall 392 and a top wall 394, and is shaped and dimensioned such that one may grasp the thickened region 380 of the belt 322 by simultaneously grasping the top wall 394 of the internal cavity 388 and the top surface 382 of the belt 322 with the opposed grippers of a conventional grasper. Since there is belt 322 material on the bottom wall 392 of the grasping pocket 380, the balloon 324 is protected during manipulation. In addition, the surgeon could come straight at the gastric band 310 and grasp the thinner region 396 on either lateral side 398 of the grasping pocket 380 to manipulate the gastric band 310.

Referring to FIGS. 10 and 11, yet another embodiment is disclosed. In accordance with this embodiment, the region just proximal of the shell member 430 along the belt 422 of the gastric band 410 is provided with an engagement member in the form of raised, necked-in region 480, in particular, at the first end 426 of the belt 422 on the side of the shell member 430 opposite the tip 444 of the first end 414 of the gastric band 410. The necked-in region 480 is generally a segment of the belt 422 with a narrowed section similar in shape to an hourglass, that is, the lateral edges 486, 488 of the belt 422 are closer together in the necked-in region 480 than along the adjacent portions of the belt 422. In utilizing this embodiment, the surgeon would come from directly above the gastric band 410 for grasping and manipulation. Although there is not belt material around the necked-in region 480 to protect the balloon 424 from the graspers, the necked-in region 480 is provided with additional height by increasing the distance between the top surface 482 and the bottom surface 484 of the belt 422 in the necked-in region 480 when compared to adjacent portions of the belt. This assists in raising the graspers and reducing the potential for balloon 424 damage.

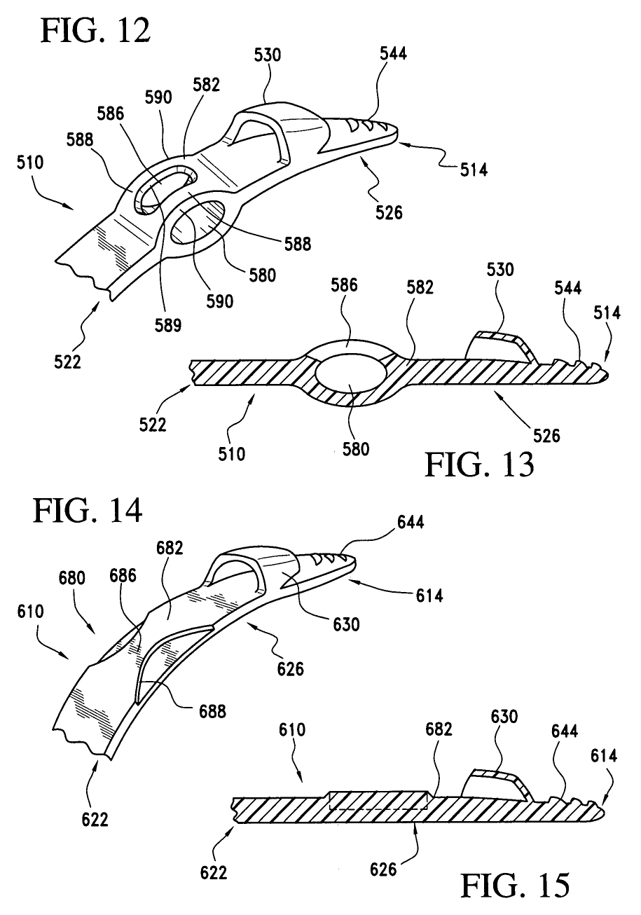

Another embodiment is disclosed with reference to FIGS. 12 and 13. This embodiment employs an engagement member in the form of a thru-hole 580 similar to that described above with reference to FIGS. 1, 2 and 3. The thru-hole 580 laterally passes through the side of the belt 522 of the gastric band 510 just proximal of the shell member 530, in particular, at the first end 526 of the belt 522 on the side of the shell member 530 opposite the tip 544 of the first end 514 of the gastric band 510. As with the prior embodiment, the thru-hole 580 allows the surgeon to slide a grasper in and manipulate the gastric band 510 from the side. In addition to the side thru-hole 580, a center hole 586 is provided along the top surface 582 of the belt 522 and in alignment with the thru-hole 580. The center hole 586 is positioned to allow access from the top of the gastric band 510. By the inclusion of the center hole 586, a surgeon may grasp either of the thin sections 588 defined by the side walls 589 of the center hole 586 and the edges 590 of the belt 522, aiding in manipulation thereof. The thru-hole 580 and center hole 586 also collapse if the surgeon grasps the entire region. Since there is belt 522 material below the thru-hole 580, that is, along the bottom surface 584 of the belt 522, the balloon 524 is protected in accordance with this concept.

Finally, and with reference to FIGS. 14 and 15, another embodiment is disclosed. In accordance with this embodiment, an engagement member in the form of a necked-in region 680 along the belt 622 of the gastric band 610 is provided just proximal of the shell member 630, in particular, at the first end 626 of the belt 622 on the side of the shell member 630 opposite the tip 644 of the first end 614 of the gastric band 610. However, and in contrast to the embodiment disclosed with reference to FIGS. 10 and 11, the necked-in region 680, that is, the lateral walls 686, 688 of the belt 622 are closer together in the necked-in region 680 than along the adjacent portions of the belt 622, only forms the top surface 682 of the belt 622 adjacent the shell member 630. As a result, there is belt 622 material below the necked-in region 680 so that the graspers will stop by hitting belt 622 material and, therefore, reduce the potential to damage the balloon 624. This embodiment may or may not incorporate a raised necked region as discussed above, although in accordance with a preferred embodiment, the necked-in region 680 is thicker than the remaining portions of the belt 622, that is, the necked-in region 680 is raised relative the top surface 682 of a remainder of the gastric belt 622.

Improved attachment of the present gastric band about a patient's stomach may be achieved through the implementation of a suture tab extender as disclosed in commonly owned U.S. patent application Ser. No. 11/364,361, entitled “Gastric Band Suture Tab Extender”, filed Mar. 1, 2006, which is incorporated herein by reference.

As those skilled in the art will certainly appreciate, a supply tube 90 is used to connect the internal cavity of the balloon 24 of the gastric band 10 with a pressurized fluid source 92, for example, a velocity port. The utilization of the supply tube 90 with a remote fluid source 92 allows for controlled inflation and deflation of the balloon 24 in a predetermined manner. The exact position of the supply tube 90 is important in that the surgeon does not want the supply tube 90 to be a visual obstruction during locking and/or other manipulation of the gastric band 10. In addition, once placement of the gastric band 10 is complete, the supply tube 90 should not cause irritation to surrounding tissue (for example, sticking directly into the liver or spleen). Surgeons also do not want to pull the supply tube 90 through a retro-gastric tunnel, since they cannot easily see if the tissue is being damaged. The supply tube 90 should also be able to act as a safe grasping location for manipulation of the gastric band 10, the supply tube 90 must not kink at the junction to the gastric band 10 and prevent fluid flow, and the supply tube 90 location should facilitate passage of the gastric band 10 through a small trocar.

In accordance with various preferred embodiments of the present invention, different tube placements may be employed as disclosed in commonly owned U.S. patent application Ser. Nos. 11/364,362, entitled “Gastric Band”, filed Mar. 1, 2006, and 11/364,363, entitled “Precurved Gastric Band”, filed Mar. 1, 2006, which are incorporated herein by reference.

Although the present invention is described for use in conjunction with gastric bands, those skilled in the art will appreciate the above invention has equal applicability to other types of implantable bands. For example, bands are used for the treatment of fecal incontinence. One such band is described in U.S. Pat. No. 6,461,292. Bands can also be used to treat urinary incontinence. One such band is described in U.S. Patent Application Publication No. 2003/0105385. Bands can also be used to treat heartburn and/or acid reflux. One such band is described in U.S. Pat. No. 6,470,892. Bands can also be used to treat impotence. One such band is described in U.S. Patent Application Publication No. 2003/0114729.

While the preferred embodiments have been shown and described, it will be understood that there is no intent to limit the invention by such disclosure, but rather, is intended to cover all modifications and alternate constructions falling within the spirit and scope of the invention.

1. A gastric band, comprising:
a gastric band body having a first end and second end, the first end and the second end being provided with a respective first latching member and a second latching member, the first end including a tip and an engagement member proximal the first latching member such that the engagement member is on a side of the first latching member opposite the tip.

2. The gastric band according to claim 1, wherein the engagement member is a thru-hole formed in the gastric band body.

3. The gastric band according to claim 2, wherein the thru-hole passes laterally through the gastric band body.

4. The gastric band according to claim 2, wherein the thru-hole includes a top surface and a bottom surface, and the top surface is narrower in its lateral dimension than the bottom surface.

5. The gastric band according to claim 1, wherein the engagement member is a shell-like member protruding from the gastric band body.

6. The gastric band according to claim 5, wherein the shell-like member includes a convex outer surface with a first end adjacent the first latching member and a second end facing away from the first latching member, and the second end of the shell-like member is open providing an access opening to a cavity of the shell-like member.

7. The gastric band according to claim 1, wherein the engagement member is a thickened region with a recess formed therein.

8. The gastric band according to claim 1, wherein the engagement member is a grasping pocket.

9. The gastric band according to claim 8, wherein the grasping pocket is formed in a thickened region in the gastric band body.

10. The gastric band according to claim 8, wherein the grasping pocket includes an internal cavity with an access opening.

11. The gastric band according to claim 10, wherein the access opening substantially faces away from the first latching member.

12. The gastric band according to claim 1, wherein the engagement member is a raised, necked-in region.

13. The gastric band according to claim 12, wherein the necked-in region includes a generally narrowed section.

14. The gastric band according to claim 13, wherein the narrowed section includes an hourglass shape.

15. The gastric band according to claim 1, wherein the engagement member is a lateral thru-hole with a center hole extending through a top surface thereof.

16. The gastric band according to claim 1, wherein the engagement member is a necked-in region wherein lateral walls of the gastric band body are closer together in the necked-in region along the top surface thereof than along the bottom surface of the gastric band body.

17. The gastric band according to claim 16, wherein the necked-in region is raised relative the top surface of a remainder of the gastric band body.
